(19) United States
(12) Patent Application Publication
(10) Pub. No.: US 2026/0321434 A1
(43) Pub. Date: Oct. 15, 2026

(54) METHODS AND SYSTEMS FOR PRESENTING RISK OF RUNNING-RELATED INJURIES PREDICTED BY MACHINE LEARNING MODELS TO A USER

(71) Applicant: RunWise AI LLC, Jacksonville, FL (US)

(72) Inventors: Bjorn G. Anderson, Jacksonville, FL (US); Gabriel K. Azar, Princeton, NJ (US)

(21) Appl. No.: 19/644,394

(22) Filed: Apr. 10, 2026

Related U.S. Application Data
(60) Provisional application No. 63/787,445, filed on Apr. 11, 2025.

(51) Int. Cl. G16H 50/30; G06N 20/00

(52) U.S. Cl. CPC G16H 50/30; G06N 20/00

(57) ABSTRACT

A computer-implemented method for presenting risk of running-related injury to a user includes providing a user interface having one or more input elements for collecting prospective training plan data from the user. The method further includes receiving historical training data for the user and constructing a runner profile based on the prospective and historical data. The runner profile is input into a trained machine learning model, which predicts a risk of musculoskeletal injury for the user. The predicted injury risk is displayed on the user interface. The user interface supports iterative adjustment of the prospective training plan, with updated risk predictions displayed in response to each adjustment. The user interface optionally includes a risk sensitivity mode input element that allows the user to calibrate prediction sensitivity based on their training context.

### CROSS REFERENCE TO RELATED APPLICATIONS

This application claims priority to U.S. provisional application No. 63/787,445, filed Apr. 11, 2025, titled “METHODS AND SYSTEMS FOR PRESENTING RISK OF RUNNING-RELATED INJURIES PREDICTED BY MACHINE LEARNING MODELS TO A USER,” the disclosure of which is incorporated herein by reference in its entirety.

### BACKGROUND

Running is a popular activity. For example, in the United States, millions of people maintain fitness by running on a regular basis. Running, however, poses a high risk of injury due to the repetitive stress on the runner's body. By some estimates, more than 50 percent of runners experience an injury each year. During time off, runners lose fitness, miss opportunities, and experience adverse physical and mental health effects. Unfortunately, preventing injuries is an extremely difficult task. In fact, conventional injury preventive measures are often either subjective (e.g., listen to your body) or rules of thumb (e.g., avoid a week-to-week mileage increase of greater than 10%, take a down week, or maintain acute to chronic ratio (ACR) less than 1.5). These conventional prevention methods are also inaccurate and have not decreased injury incidence. Moreover, researchers have not yet uncovered any predictive characteristics (e.g., strength, flexibility, biomechanics, injury history, etc.) to identify which runners are likely to get injured and/or why so. See Hutchinson, Alex, The Elusive Art of Predicting Injuries, Outside Online.com, published May 7, 2021, https://www.outsideonline.com/2423442/running-injuries-prediction-research (accessed May 8, 2021).

The lack of injury prevention solutions is also a problem for coaching platforms, including virtual, artificial intelligence(AI)-powered platforms. For example, recent reporting has highlighted that AI-powered fitness platforms often push athletes past their physical limits because current models rely on quantitative training data, such as speed and distance, to adjust goals but lack insight into important to injury prevention information, such as how the athlete feels or whether the athlete is experiencing symptoms of injury. See Nguyen, Nicole, Runna Pushed Some Runners Into Injuries. The Virtual-Coach App is Now Easing Up, Wall Street Journal, published Feb. 8, 2026, https://www.wsj.com/tech/personal-tech/runna-app-race-training-09c1a723 (accessed Feb. 10, 2026).

FIG. 7 illustrates a graph illustrating cumulative training load (CTL) 702 (sometimes referred to as “fitness”), acute training load (ATL) 704 (sometimes referred to as “fatigue”), and training stress balance (TSB) 706 (sometimes referred to as “form”) according to existing techniques. The plots of CTL, ATL, and TSB are based on the impulse response model developed by Dr. Eric Banister in 1975, which is widely used by athletes and coaches to model fitness-induced changes. The CTL and ATL components are modeled as exponentially weighted moving averages of daily training dose over longer (e.g., 42 days) and shorter (e.g., 7 days) periods of time, respectively. CTL is intended to represent the athlete's positive training adaptation, while ATL is intended to represent the negative impacts of training. TSB is the difference between CTL and ATL. The objective is to maximize CTL with TSB of about zero (or slightly negative) at the time of a goal event, meaning the athlete is fit and well rested when it matters most. The graph does not include any visual indication of injury risk. Instead, athletes and coaches understand trends in the plots of CTL, ATL, and TSB and also that overtraining may occur when ATL exceeds CTL (e.g., TSB<<0) by “too much” or for “too long.” Neither “too much” nor “too long” is quantified by the plots or the equations. Overtraining is therefore associated with unquantifiable risks of injury, sickness, and/or performance decline. The plots of FIG. 7 do not offer any means to determine which of injury, sickness, or poor performance will occur, or when such undesired outcome will occur. Like injury risk, there is no visual indication of when the athlete is in an overtrained state.

FIG. 8 illustrates a graphical display of acute to chronic ratio (ACR) according to an existing technique. ACR is a ratio of 1-week mileage (total mileage last 7 days) to 4-week average mileage (weekly average mileage last 28 days). The objective is to maintain ACR less than 1.5 to minimize risk of injury since ACR>1.5 has been shown to associate with increased risk of injury. In the graphical display of FIG. 8, green shading (labeled 802) corresponds to 0.8<ACR<1.3, orange shading (labeled 804) corresponds to 1.3<ACR<1.5, and red shading (labeled 806) corresponds to ACR>1.5. The graphical display allows the athlete to enter training data and view the projected effect on the ACR curve, for example by adjusting planned runs to keep the training line 810 in the green zone. However, ACR is merely a ratio of acute to chronic load (i.e. a heuristic or rule of thumb based on mileage). Even if ACR is interpreted as a risk score, it is neither derived from a trained machine learning model nor presented in a manner that reflects the complex, multidimensional patterns in training data that contribute to injury risk. ACR does not allow the athlete to toe the line between improving fitness and avoiding injury. It is overly simple and coarse.

There is therefore a need in the art for tools to display predicted running-related injury risk.

### SUMMARY

Existing tools for monitoring running training load fail to provide runners with a quantitative, visually actionable display of injury risk. Conventional graphical displays, such as impulse response model plots of cumulative training load, acute training load, and training stress balance, present fitness and fatigue trends but include no indication of injury risk whatsoever. Similarly, acute-to-chronic ratio displays present a simple heuristic ratio of recent to historical mileage, which, even if interpreted as a proxy for injury risk, is a coarse rule of thumb that cannot identify which runners are likely to be injured or when. Neither approach allows a runner to contextualize predicted injury risk within a longitudinal record of training activity, assess the injury implications of a prospective training plan before executing it, or calibrate the sensitivity of risk warnings to their individual training context and goals.

The present disclosure addresses the above deficiencies by providing a user interface that presents a machine learning-derived injury risk to the user in a manner that is integrated with the user's training journal. In some aspects, the techniques described herein relate to a computer-implemented method for presenting risk of running-related injury to a user, including: receiving running-related data for a user; creating a runner profile for the user based on the running-related data; inputting the runner profile into a trained machine learning model; predicting, using the trained machine learning model, a risk of musculoskeletal injury for the user; and outputting the risk of musculoskeletal injury, wherein the risk of musculoskeletal injury is configured for display on a user interface of an application, and wherein the user interface comprises a journal view of the user's training data.

In some aspects, the user's training data includes retrospective training data and prospective training plan data.

In some aspects, the method further includes displaying, on the user interface, the risk of musculoskeletal injury.

In some aspects, the running-related data is received from an application program interface (API) of the application, and wherein the risk of musculoskeletal injury is output to the API of the application.

In some aspects, the techniques described herein relate to a computer-implemented method for presenting risk of running-related injury to a user, including: receiving running-related data for a user; creating a runner profile for the user based on the running-related data; inputting the runner profile into a trained machine learning model; predicting, using the trained machine learning model, a risk of musculoskeletal injury for the user; and displaying, on a user interface, the risk of musculoskeletal injury, wherein the user interface comprises a journal view of the user's training data.

Alternatively or additionally, the present disclosure addresses the above deficiencies by providing a user interface that collects prospective training plan data, combines it with historical training data, and presents a machine learning-derived injury risk to the user in a manner that is integrated with the user's training journal, and optionally supports iterative adjustment of planned training. In some aspects, the techniques described herein relate to a computer-implemented method for presenting risk of running-related injury to a user, including: providing a user interface including one or more input elements for collecting first running-related data for a user; receiving the first running-related data, wherein the first running-related data is entered at the user interface; receiving, from an application, second running related data for the user; creating a runner profile for the user based on the first and second running-related data; inputting the runner profile into a trained machine learning model; predicting, using the trained machine learning model, a risk of musculoskeletal injury for the user; and displaying, on the user interface, the risk of musculoskeletal injury.

In some aspects, the user interface further includes one or more display elements for displaying the risk of musculoskeletal injury.

In some aspects, the one or more input elements include a total volume field and an intensity field. Optionally, the one or more input elements further include a maximum volume field. Optionally, the one or more input elements further include a consistency field. Optionally, the one or more input elements further include a dynamic field or a physiological field.

In some aspects, the user interface further includes an input element for collecting a risk sensitivity mode, and the method further includes receiving the risk sensitivity mode, wherein the risk sensitivity mode is entered at the user interface.

In some aspects, the method further includes generating a report that summarizes the user's training data.

In some aspects, the method further includes displaying, on the user interface, the report.

In some aspects, the method further includes transmitting the report to the user.

In some aspects, the report includes the risk of musculoskeletal injury.

In some aspects, the user interface further includes an input element for collecting an indication of an injury, and the method further includes receiving the indication, wherein the indication is entered at the user interface.

In some aspects, the indication includes a type, a description, or a location of the injury.

In some aspects, the method further includes: inputting, into a language model, one or more of the runner profile and the indication; generating, using the language model, a recommendation about the injury; and outputting the recommendation, wherein the recommendation is configured for display on the user interface.

In some aspects, the method further includes: receiving a user input including a question concerning the injury; processing, by a language model, the user input to extract key information; generating, by the language model, a response based on the key information; and outputting the response, wherein the response is configured for display on the user interface.

Alternatively or additionally, the present disclosure addresses the above deficiencies by providing a user interface that enables user-driven calibration of predictive model sensitivity based on the asymmetric costs of missed warnings versus false alarms. In some aspects, the techniques described herein relate to a computer-implemented method, including: receiving running-related data for a user; creating a runner profile for the user based on the running-related data; receiving a risk sensitivity mode; selecting a trained machine learning model or one or more thresholds for the trained machine learning model based on the risk sensitivity mode; inputting the runner profile into a trained machine learning model; and predicting, using the trained machine learning model, a risk of musculoskeletal injury for the user. Optionally, the method further includes displaying, on a user interface, the risk of musculoskeletal injury.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example dataset comprising running-related data.

FIG. 2A is a flowchart of an example method for presenting risk of running-related injury to a user according to an implementation described herein. FIG. 2B is a flowchart of another example method for presenting risk of running-related injury to a user according to an implementation described herein.

FIGS. 3A-3D illustrate example user interfaces with a journal view of training data according to implementations described herein. FIG. 3A illustrates an example training calendar view. FIG. 3B illustrates an example training log view. FIG. 3C illustrates another example training calendar view. FIG. 3D illustrates yet another example training calendar view.

FIGS. 4A-4B illustrate example user interfaces with input and display elements according to implementations described herein. Each of FIGS. 4A and 4B illustrates a user interface for planning and injury risk management.

FIGS. 5A-5B illustrate other example user interfaces with input and display elements according to implementations described herein. FIG. 5A illustrates a user interface for assessing a training plan. FIG. 5B illustrates a user interface for displaying results of such assessment.

FIG. 6 is an example computing device.

FIG. 7 a graph illustrating cumulative training load, acute training load, and training stress balance according to existing techniques.

FIG. 8 is a graphical display of acute chronic ratio according to an existing technique.

FIG. 9 is a table illustrating classification thresholds for a predictive model according to an implementation described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately” when referring to a measurable value such as an amount, a percentage, and the like, is meant to encompass variations of ±20%, 10%, 5%, or +1% from the measurable value.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

As used herein, musculoskeletal injuries affect a runner's bones, joints, or soft tissues such as muscles, tendons, ligaments, or other connective tissue. Running-related injuries include, but are not limited to, those affecting the feet, knees, upper or lower legs, hips, pelvis, or groin. Example running-related musculoskeletal injuries include, but are not limited to, stress fractures, tendonitis, plantar fasciitis, iliotibial (IT) band syndrome, strains, and sprains. Additionally, this disclosure contemplates that a musculoskeletal injury forces a runner to rest (not run) for an extended period of time (e.g., from 3-5 days or longer such as several weeks, months, or even longer). Thus, as used herein, a running-related injury results in a runner taking 3 or more consecutive days of rest. Optionally, a running-related injury results in a runner taking at least 5 consecutive days of rest.

This disclosure contemplates that the running-related datasets described herein can be obtained from a runner's log, e.g., the record used to track running-related information such as mileage, running duration, physiological data, environmental conditions, injuries, or other information related to running. Optionally, the runner's log is maintained in an electronic medium. For example, Internet-based services for tracking fitness data are in common use by runners. Example Internet-based services include, but are not limited to, the STRAVA app and website of Strava, Inc. of San Francisco, California, GARMIN CONNECT app and website of Garmin International of Olathe, Kansas, the TRAININGPEAKS app and website of TrainingPeaks, LLC of Louisville, Colorado, and the FINAL SURGE app and website of Final Surge LLC of Raleigh, North Carolina. It should be understood that the STRAVA, GARMIN CONNECT, TRAININGPEAKS, and FINAL SURGE apps and websites are provided only as example Internet-based services. This disclosure contemplates that other electronic and/or Internet-based services may be used to track running-related data.

Internet-based services maintain a vast amount of running-related data for a plurality of runners. For example, the STRAVA app and website had approximately 150 million users in 2025. Running-related data includes, but is not limited to, global positioning system (GPS) route data; mileage; duration; pace; speed; sensor data (e.g., heart rate monitor, accelerometer, etc.); dynamic data (e.g., cadence, stride length, vertical oscillations, ground contact time); perceived effort; and free-form comments. Running-related data for individual sessions can be stored in XML format files such as GPX (GPS Exchange) or TCX (Training Center XML) file types or binary format files such as FIT (flexible and interoperable data transfer) file types. Running-related data for multiple sessions can be stored as structured data in a database. Such running-related data is primarily measured using a device, for example, a running watch, fitness tracker, or mobile phone. These devices include built-in location service such as GPS and, optionally, built-in or external sensors. An example running watch is the GARMIN FORERUNNER watch of Garmin International of Olathe, Kansas. It should be understood that the GARMIN FORERUNNER watch is provided only as an example. This disclosure contemplates that other devices, including but not limited to the APPLE Watch of Apple Inc. of Cupertino, California, may be used to measure running-related data. Alternatively, running-related data may be measured using other dedicated running devices such as footpods, smart insoles, or smart shoes. Alternatively or additionally, running-related data may be entered or altered by the runner.

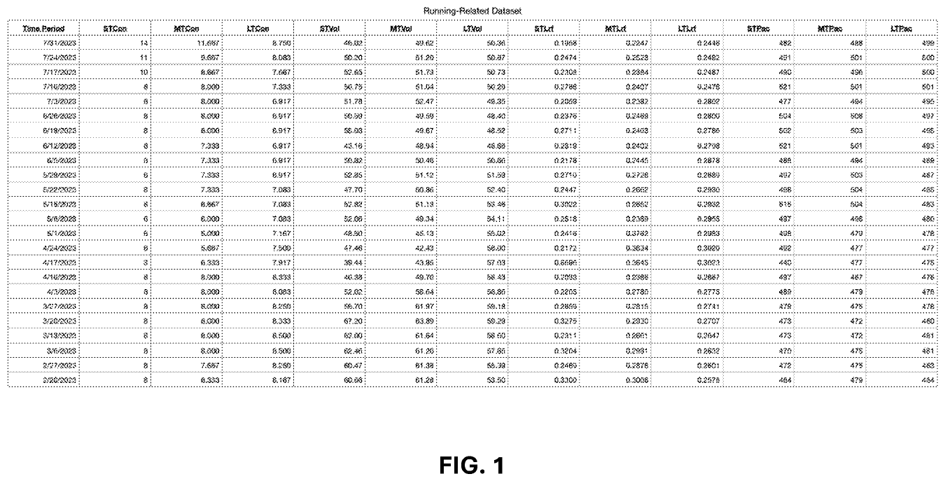

Referring now to FIG. 1, an example dataset comprising running-related data is shown. The running data was obtained from an electronic runner's log, and metrics (e.g., volume, intensity, long run fraction, consistency) were calculated as described below using the Python programming language. The example dataset shown in FIG. 1 includes 24 rows of running-related data, which is grouped by week. Accordingly, each row corresponds to a week between 2/20/2023 and 7/31/2023, and each column corresponds to a metric. It should be understood that the number of weeks of data in FIG. 1 (i.e., 24 weeks) is provided only as an example. In some implementations, the dataset may include more than 24 weeks of running-related data, e.g., 25, 26, 27, 28, 29, 30, 31, 32, 33, 34, 35, 36, or more weeks of running-related data. Optionally, the dataset may include 36 weeks of running-related data. Optionally, the dataset may include 52 weeks of running-related data. Alternatively, the dataset may include less than 24 weeks of running-related data, e.g., 23, 22, 21, 20, 19, 18, or less weeks of running-related data. Optionally, the dataset may include 18 weeks of running-related data. It should also be understood that the running-related data may include more or less metrics than shown in FIG. 1.

As described below, metrics are provided for short-term, medium-term, and long-term periods. As used herein, a short-term period represents a training period. A training period can optionally be a 7 day period (e.g., a calendar week). It should be understood that a training period may be more or less than 7 days (e.g., a 10-day or 5-day period). It should also be understood that the training period length can be selected by a runner. As used herein, a medium-term period includes a plurality of training periods. The number of training periods in a medium-term period is selected to create metrics representing the transient fitness level of and stress on the runner. For example, the medium-term period can be a 2-8 week period (i.e., 2-8, 7-day training periods). Optionally, the medium-term period can be a 3 week period (i.e., three, 7-day training periods). It should be understood that 2-8 weeks is only provided as an example. As used herein, a long-term period includes a plurality of training periods, which is greater than the number of training periods of the medium-term period. The number of training periods in a long-term period is selected to create metrics representing the base fitness level of and stress on the runner. For example, the long-term period can be a 10-18 week period (i.e., 10-18, 7-day training periods). Optionally, the long-term period can be a 12 week period (i.e., twelve, 7-day training periods). It should be understood that 10-18 weeks is only provided as an example.

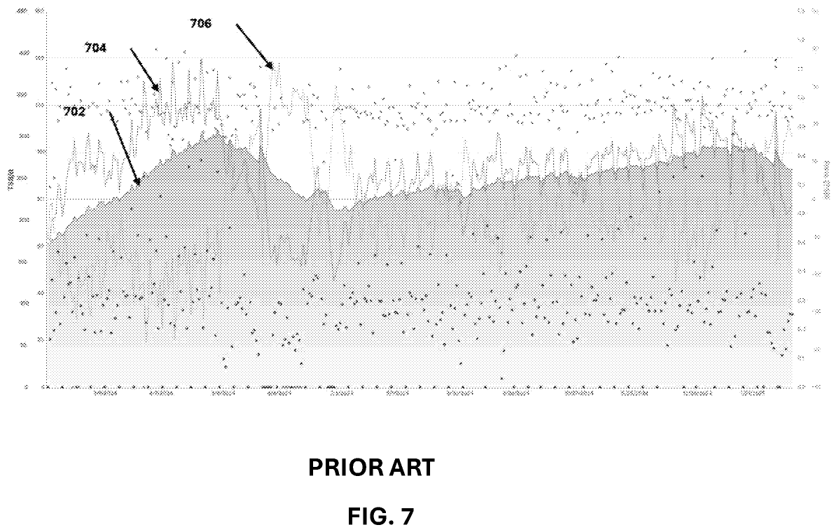

In some implementations, the short-term period is used to characterize the runner's acute training load (sometimes referred to as the runner's “fatigue” level), and the long-term period is used to characterize the runner's chronic training load (sometimes referred to as the runner's “fitness” level). Acute and chronic training loads have been used in the art to assess training-induced physiological changes experienced by runners (see e.g., FIG. 7). For example, according to some methodologies, the short-term period is optionally 7 days (i.e., 1 week), and the long-term period is optionally 42 days (i.e., 6 weeks). It should be understood that 7 days and 42 days are provided only as example lengths for the short-term and long-term periods, respectively. This disclosure contemplates that the short-term and long-term periods can have other lengths.

The dataset includes volume metrics. Volume metrics include, but are not limited to, a daily volume metric, a short-term volume metric, a medium-term volume metric, and a long-term volume metric. Optionally, in some implementations as shown in FIG. 1, the volume metrics includes a short-term volume metric (STVol in FIG. 1), a medium-term volume metric (MTVol in FIG. 1), and a long-term volume metric (LTVol in FIG. 1). This disclosure contemplates that a volume metric is a measure of running time or duration (e.g., hours, minutes, seconds) and/or running distance (e.g., miles, kilometers). In FIG. 1, volume is a distance (miles). Additionally, this disclosure contemplates that the volume metrics can be obtained (e.g., received, downloaded, etc.) and/or derived from the electronic runner's log described above. As used herein, a daily volume metric is a 1-day cumulative run length (e.g., daily total), which can optionally include one or more runs. As used herein, a short-term volume metric is the cumulative run length during a training period. Additionally, as described above, a training period can optionally be a 7 day period (e.g., a calendar week). It should be understood that a training period may be more or less than 7 days (e.g., a 10-day or 5-day period). As used herein, a medium-term volume metric is an average cumulative run length over a plurality of training periods, for example, the average training period (e.g., weekly) run length over a 2-8 week period. It should be understood that 2-8 weeks is only provided as an example medium-term period. As used herein, a long-term volume metric is an average cumulative run length over a plurality of training periods, for example, the average training period (e.g., weekly) run length over a 10-18 week period. It should be understood that 10-18 weeks is only provided as an example long-term period. The short-, medium-, and long-term volume metrics represent cumulative run lengths over progressively longer periods of time. Additionally, as described above, run length can be measured by a duration and/or a distance.

The table in FIG. 1 illustrates short-term, medium-term, and long-term volume metrics. For example, the table includes short-term volume metrics (“STVol”), medium-term volume metrics (“MTVol”), and long-term volume metrics (“LTVol”) for an example runner during twenty four (24) consecutive weeks in 2023 (i.e., weeks of February 20^{th} through July 31^{st}). In FIG. 1, the short-term, medium-term, and long-term periods are 1, 3, and 12 weeks, respectively. As described above, these lengths are provided only as examples. Additionally, it should be understood that the metrics shown in FIG. 1 were obtained from and/or calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

The dataset also includes intensity metrics. Intensity metrics include, but are not limited to, a daily intensity metric, a short-term intensity metric, a medium-term intensity metric, and a long-term intensity metric. Optionally, in some implementations as shown in FIG. 1, the intensity metrics includes a short-term intensity metric (STPac in FIG. 1), a medium-term intensity metric (MTPac in FIG. 1), and a long-term intensity metric (LTPac in FIG. 1). This disclosure contemplates that an intensity metric is a running pace or running speed. Pace is measured as a time per distance unit (e.g., minutes per mile or minutes per kilometer). Speed is measured as distance per unit time (e.g., miles per hour or kilometers per hour). In FIG. 1, intensity is a pace (seconds per mile). Additionally, this disclosure contemplates that the intensity metrics can be obtained (e.g., received, downloaded, etc.) and/or derived from the electronic runner's log described above. As used herein, a daily intensity metric is a 1-day average intensity (e.g., pace or speed). As used herein, a short-term intensity metric is the average intensity (e.g., pace or speed) during a training period. As used herein, a medium-term intensity metric is the average intensity (e.g., pace or speed) over a plurality of training periods. As used herein, a long-term intensity metric is the average intensity (e.g., pace or speed) over a plurality of training periods. The daily, short-, medium-, and long-term intensity metrics represent average intensity over progressively longer periods of time. Additionally, as described above, run intensity can be measured by pace or speed.

The table in FIG. 1 illustrates short-term, medium-term, and long-term intensity metrics. For example, the table includes short-term volume metrics (“STPac”), medium-term volume metrics (“MTPac”), and long-term volume metrics (“LTPac”) for an example runner during twenty four (24) consecutive weeks in 2023 (i.e., weeks of February 20^{th} through July 31^{st}). In FIG. 1, the short-term, medium-term, and long-term periods are 1, 3, and 12 weeks, respectively. As described above, these lengths are provided only as examples. Additionally, it should be understood that the metrics shown in FIG. 1 were obtained from and/or calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

The dataset also includes long run fraction metrics. Long run fraction metrics include, but are not limited to, one or more of a short-term long run fraction metric (STLrf in FIG. 1), a medium-term long run fraction metric (MTLrf in FIG. 1), and a long-term long run fraction metric (LTLrf in FIG. 1). In FIG. 1, the long run fraction metric represents a long run volume (miles) divided by a training period volume (miles). For example, if a runner's longest run during a 7-day training period is 10 miles and the runner's total mileage during the 7-day training period is 50 miles, the long run fraction metric is 0.2. Additionally, this disclosure contemplates that the long run fraction metrics can be obtained (e.g., received, downloaded, etc.) and/or derived from the electronic runner's log described above. As used herein, a short-term long run fraction metric is a long run volume divided by total volume during a training period. For example, the short-term long run fraction metric is 0.2 when a runner's longest run is 10 miles during 7-day training period where total mileage is 50 miles. As used herein, a medium-term long run fraction metric is the average long run fraction metric over a plurality of training periods. For example, if a runner's long run fraction is 0.2, 0.3, and 0.4 during each of three consecutive 7-day training periods, respectively, then the medium-term long run fraction metric is 0.3. As used herein, a long-term long run fraction metric is the average long run fraction metric over a plurality of training periods. For example, if a runner's long run fraction is 0.2, 0.3, 0.4, 0.25, 0.3, 0.25, 0.2, 0.2, 0.4, 0.3, 0.25, and 0.2 during each of twelve consecutive 7-day training periods, respectively, then the long-term long run fraction metric is 0.27. The short-, medium-, and long-term long run fraction metrics capture the training period-to-training period fractional contribution of a runner's longest run to total volume over progressively longer periods of time. This disclosure contemplates that patterns predictive of injury risk are present in the volume, intensity, and long run fraction metrics found in running-related data.

The table in FIG. 1 illustrates short-term, medium-term, and long-term long run fraction metrics. For example, the table includes short-term volume metrics (“STLrf”), medium-term volume metrics (“MTLrf”), and long-term volume metrics (“LTLrf”) for an example runner during twenty four (24) consecutive weeks in 2023 (i.e., weeks of February 20^{th} through July 31^{st}). In FIG. 1, the short-term, medium-term, and long-term periods are 1, 3, and 12 weeks, respectively. As described above, these lengths are provided only as examples. Additionally, it should be understood that the metrics shown in FIG. 1 were calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

The dataset also includes consistency metrics. Consistency metrics include, but are not limited to, a short-term consistency metric (STCon in FIG. 1), a medium-term consistency metric (MTCon in FIG. 1), and a long-term consistency metric (LTCon in FIG. 1). This disclosure contemplates that a consistency metric represents a number of running days (or number of runs) during the short-term, medium-term, and/or long-term periods. In FIG. 1, consistency is a raw number of runs during a training period. Additionally, this disclosure contemplates that the consistency metrics can be obtained (e.g., received, downloaded, etc.) and/or derived from the electronic runner's log described above. As used herein, a short-term consistency metric is the number of running days or raw number of runs during a training period. For example, if a runner ran everyday Monday through Friday during a 7-day training period, then the short-term consistency metric is 5. As used herein, a medium-term consistency metric is the average consistency over a plurality of training periods. For example, if a runner ran 5, 6, and 7 days during each of three consecutive 7-day training periods, respectively, then the medium-term consistency metric is 6. As used herein, a long-term intensity metric is the average consistency over a plurality of training periods. For example, if a runner ran 5, 6, 7, 0, 1, 1, 5, 6, 7, 4, 2, and 4 days during each of twelve consecutive 7-day training periods, respectively, then the long-term consistency metric is 4. The short-, medium-, and long-term consistency metrics represent a runner's training period-to-training period consistency over progressively longer periods of time. Additionally, as described above, consistency can be measured by a number of running days or raw number of runs. This disclosure contemplates that patterns predictive of injury risk are present in the volume, intensity, long run fraction, and consistency metrics found in running-related data.

The table in FIG. 1 illustrates short-term, medium-term, and long-term consistency metrics. For example, the table includes short-term volume metrics (“STCon”), medium-term volume metrics (“MTCon”), and long-term volume metrics (“LTCon”) for an example runner during twenty four (24) consecutive weeks in 2023 (i.e., weeks of February 20^{th} through July 31^{st}). In FIG. 1, the short-term, medium-term, and long-term periods are 1, 3, and 12 weeks, respectively. As described above, these lengths are provided only as examples. Additionally, it should be understood that the metrics shown in FIG. 1 were obtained from and/or calculated from data included in the example runner's electronic log. This disclosure contemplates that short-term, medium-term, and/or long-term metrics can be calculated using any tools known in the art including, but not limited to a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit.

Alternatively or additionally, the dataset can optionally include at least one variability metric. Variability metrics include, but are not limited to, a short-term variability metric, a medium-term variability metric, and a long-term variability metric. This disclosure contemplates that a variability metric represents a number of high-intensity running days (or number of high-intensity runs) during the short-term, medium-term, and/or long-term periods. As used herein, a high-intensity run is a run requiring greater than ordinary effort by a runner. For example, a workout and a race are considered high intensity runs. Additionally, this disclosure contemplates that the variability metrics can be obtained (e.g., received, downloaded, etc.) and/or derived from the electronic runner's log described above. As used herein, a short-term variability metric is the number of high-intensity running days or raw number of high-intensity runs during a training period. For example, if a runner ran with high-intensity (e.g., workout, race, etc.) twice during a 7-day training period, then the short-term variability metric is 2. As used herein, a medium-term variability metric is the average variability over a plurality of training periods. For example, if a runner ran with high intensity (e.g., workout, race, etc.) 2, 1, and 1 days during each of three consecutive 7-day training periods, respectively, then the medium-term variability metric is 1.33. As used herein, a long-term variability metric is the average variability over a plurality of training periods. For example, if a runner ran with high intensity (e.g., workout, race, etc.) 2, 1, 1, 0, 0, 1, 1, 2, 2, 0, 1, and 1 days during each of twelve consecutive 7-day training periods, respectively, then the long-term variability metric is 1. The short-, medium-, and long-term variability metrics capture training period-to-training period high-intensity efforts over progressively longer periods of time. Additionally, as described above, variability can be measured by a number of high-intensity running days or raw number of high-intensity runs. This disclosure contemplates that patterns predictive of injury risk are present in the volume, intensity, long run fraction, consistency, and variability metrics or combinations thereof found in running-related data.

Alternatively or additionally, the dataset can optionally include at least one dynamic metric. Dynamic metrics include, but are not limited to, a daily dynamic metric, a short-term dynamic metric, a medium-term dynamic metric, and a long-term dynamic metric. Optionally, in some implementations, the dynamic metrics includes a short-term dynamic metric, a medium-term dynamic metric, and a long-term dynamic metric. This disclosure contemplates that a dynamic metric defines an aspect of a runner's motion. Dynamic metrics can be derived from sensor data such as accelerometer or inertial sensor data. Example dynamic metrics include, but are not limited to, cadence, stride length, ground contact time, or vertical oscillations. Additionally, this disclosure contemplates that the dynamic metrics can be obtained (e.g., received, downloaded, etc.) and/or derived from the electronic runner's log described above. As used herein, a daily dynamic metric is a 1-day average dynamic metric such as cadence, stride length, ground contact time, or vertical oscillations. As used herein, a short-term dynamic metric is the average dynamic metric such as cadence, stride length, ground contact time, or vertical oscillations during a training period. As used herein, a medium-term dynamic metric is the average dynamic metric such cadence, stride length, ground contact time, or vertical oscillations over a plurality of training periods. As used herein, a long-term dynamic metric is the average dynamic metric such as cadence, stride length, ground contact time, or vertical oscillations over a plurality of training periods. The daily, short-, medium-, and long-term dynamic metrics represent average dynamic metrics such as cadence, stride length, ground contact time, or vertical oscillations over progressively longer periods of time. This disclosure contemplates that patterns predictive of injury risk are present in the combination of volume, intensity, long run fraction, consistency, variability, and dynamic metrics or combinations thereof found in running-related data.

Alternatively or additionally, the dataset can optionally include a physiological metric. Physiological metrics include, but are not limited to, a daily physiological metric, a short-term physiological metric, a medium-term physiological metric, and a long-term physiological metric. Optionally, in some implementations, the physiological metrics includes a short-term physiological metric, a medium-term physiological metric, and a long-term physiological metric. Physiological metrics include, but are not limited to, heart rate data, oxygen saturation data, or VO₂ max data, for example. Optionally, the heart rate data is average heart rate or heart rate variability (HRV). Additionally, this disclosure contemplates that the physiological metrics can be obtained (e.g., received, downloaded, etc.) and/or derived from the electronic runner's log described above. As used herein, a daily physiological metric is a 1-day average physiological metric such as heart rate during a run. As used herein, a short-term physiological metric is the average physiological metric such as running heart rate during a training period. As used herein, a medium-term physiological metric is the average physiological metric such as running heart rate over a plurality of training periods. As used herein, a long-term physiological metric is the average physiological metric such as running heart rate over a plurality of training periods. The daily, short-, medium-, and long-term physiological metrics represent the average physiological metric over progressively longer periods of time. This disclosure contemplates that patterns predictive of injury risk are present in the volume, intensity, long run fraction, consistency, variability, dynamic, and physiological metrics or combinations thereof found in running-related data.

As described above, existing training load tools present metrics, such as cumulative training load, acute training load, training stress balance, and acute-to-chronic ratio, through graphical displays that provide no indication of injury risk. In an implementation, the present disclosure addresses this limitation by providing a method that receives running-related data, constructs a runner profile, and inputs that profile into a trained machine learning model to predict risk of musculoskeletal injury, which is then output for display on a user interface. A feature of the disclosed method is the integration of the predicted injury risk into a journal view of the user's training data, situating the machine learning-derived risk output directly within the runner's longitudinal training record. This integration provides a technical improvement over prior art training load displays by enabling a user to correlate predicted injury risk with the specific training activities and patterns that contributed to it, which is not possible with existing visualizations. The journal view further supports iterative training plan adjustment by providing the user with a unified display context in which both historical training data and current injury risk prediction are simultaneously accessible.

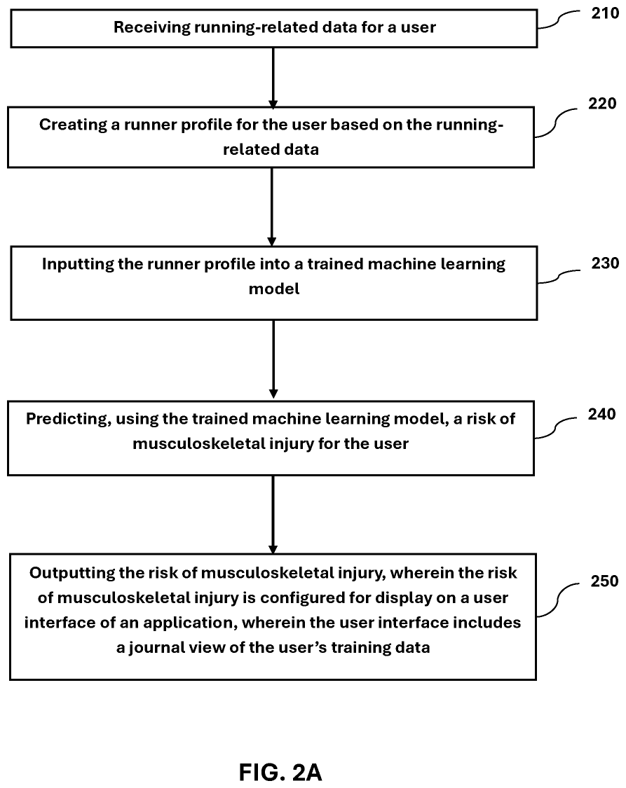

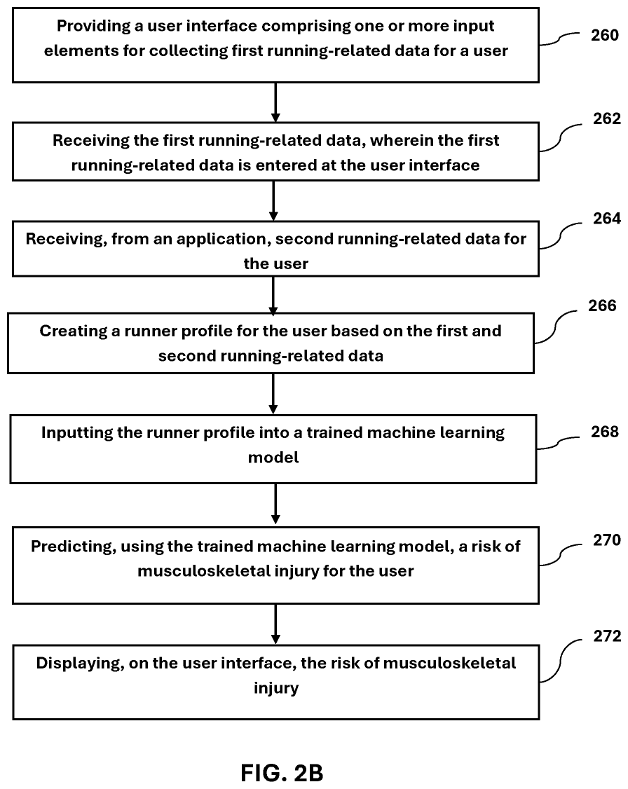

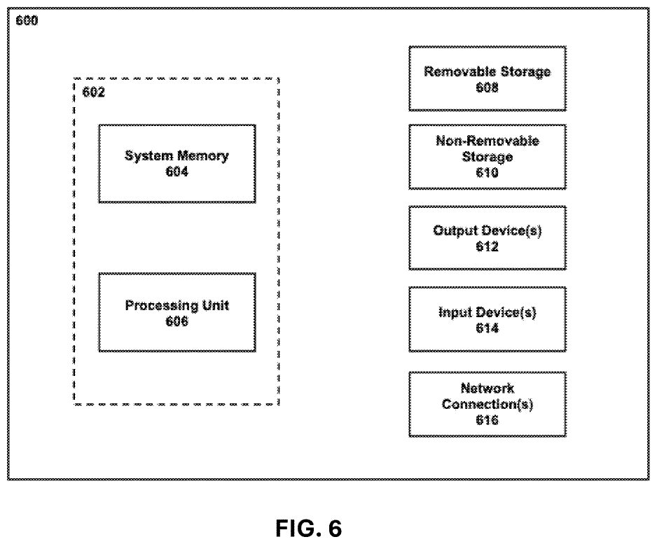

FIG. 2A is a flowchart of an example method for presenting risk of running-related injury to a user. This disclosure contemplates that the method of FIG. 2A can be performed using one or more computing devices, e.g., computing device 600 shown in FIG. 6. At step 210, the method includes receiving running-related data for a user. Running-related data can include, but is not limited to, volume, intensity, and consistency data for the user. Example running-related data is discussed above with regard to FIG. 1. Optionally, the running-related data includes one or more of variability, dynamic, and physiological data discussed above with regard to FIG. 1. Optionally, the running-related data is received from an application. For example, the application can optionally be a fitness tracking platform. Non-limiting fitness tracking platforms include, but are not limited to, the STRAVA app and website of Strava, Inc. of San Francisco, California, the GARMIN CONNECT app and website of Garmin International of Olathe, Kansas, the TRAININGPEAKS app and website of TrainingPeaks, LLC of Louisville, Colorado, and the FINAL SURGE app and website of Final Surge LLC of Raleigh, North Carolina. Optionally, the running-related data is received through an application programming interface (API) of the fitness tracking platform (sometimes referred to herein as a “connected application”). Alternatively or additionally, the application can optionally be a running-related application such as the RUNWISE injury prediction platform of RunWise AI LLC of Jacksonville, Florida. As described herein, such a running-related application can provide a user interface for collecting all or a portion of the running-related data. For example, in some implementations, historical running data can be received from a fitness tracking platform and additional data such as data associated with a runner's future training plans can be received from the running-related application (e.g. entered via the user interface).

At step 220, the method includes creating a runner profile for the user based on the running-related data. The runner profile can include one or more of the volume, intensity, consistency, long run fraction, variability, dynamic, and physiological metrics shown and/or discussed above with regard to FIG. 1. As used herein, the runner profile includes one or more “features” that are input into a machine learning model. For example, creating the runner profile can include obtaining and/or calculating one or more short-term metrics discussed above with regard to FIG. 1, e.g., the short-term volume metric, the short-term intensity metric, the short-term consistency metric, and/or the short-term long run fraction metric. Additionally, creating the runner profile can including calculating one or more of the medium-term and long-term metrics discussed above with regard to FIG. 1. Example short-term, medium-term, and long-term metrics, as well as examples methods for calculations (if needed), are discussed above. The metrics of the runner profile serve as the “features” for the machine learning analysis.

In some implementations, the runner profile includes at least one volume metric and at least one intensity metric. Additionally, in some implementations, the runner profile optionally further includes one or more of at least one consistency metric, at least one long run fraction metric, at least one variability metric, at least one dynamic metric, and/or at least one physiological metric. The runner profile may be associated with the most-recent training period (e.g., the previous 7-day period) for a retrospective analysis. Alternatively, the runner profile may be associated with a future training period (e.g., the next 7-day period) for a prospective analysis. In one implementation, the runner profile includes at least one volume metric and at least one intensity metric. Optionally, the runner profile further includes at least one long run fraction metric. Optionally, the runner profile further includes one or more of at least one consistency metric, at least one variability metric, at least one dynamic metric, or at least one physiological metric. For example, in another implementation, the runner profile includes short-, medium-, and long-term volume metrics, short-, medium-, and long-term intensity metrics, and short-, medium-, and long-term long run fraction metrics (i.e. a 9 features). In yet another implementation, the runner profile includes short-, medium-, and long-term consistency metrics, short-, medium-, and long-term volume metrics, short-, medium-, and long-term intensity metrics, and short-, medium-, and long-term long run fraction metrics (i.e. a 12 features).

Additionally, the runner profile can be a scaled runner profile or feature tensor. The runner profile and/or the running-related dataset can be scaled based on one or more characteristics of the running-related dataset. The dataset shown in FIG. 1 is unscaled. As used herein, a scaled runner profile or dataset has undergone a scaling process, where the values of the data have been transformed to a specific range or distribution. Scaling is a preprocessing step in machine learning to ensure that all features have a similar scale and to prevent certain features from dominating the learning process due to their larger values. Two example scaling processes are standardization (also known as z-score normalization) and normalization (also known as min-max scaling). This disclosure contemplates performing data scaling using tools known in the art including, but not limited to using a spreadsheet (e.g., MICROSOFT EXCEL spreadsheets of Microsoft Corp. of Redmond, WA), a computer program or application (e.g., MATLAB platform of MathWorks Corp. of Natick, MA), or a programming language (e.g., Python) library or toolkit. It should be understood that standardization and normalization are provided only as example scaling techniques. This disclosure contemplates using other scaling techniques with the implementations described herein. Scaling of feature tensors is described in U.S. Patent App. Pub. No. 2025/0054635, published Feb. 13, 2025, titled “MACHINE LEARNING-BASED METHODS AND SYSTEMS FOR PREDICTING RUNNING-RELATED INJURIES,” the disclosure of which is incorporated herein by reference in its entirety.

An example unscaled runner profile and scaled runner profile (also referred to herein as “feature tensor”) extracted from the data associated with the week of Jul. 31, 2023 in the example running-related dataset of FIG. 1 is provided below. The feature tensor has been standardized based on characteristics of the dataset, e.g. using its mean and standard deviation. The unscaled/scaled runner profile includes (in order) short-term, medium-term, and long-term metrics for consistency (STCon, MTCon, LTCon), volume (STVol, MTVoI, LTVol), long run fraction (STLrf, MTLrf, LTLrf), and intensity (STPac, MTPac, LTPac).

### Unscaled Runner Profile

[[1.40000000e+01 1.16666667e+01 8.75000000e+00 4.60200000e+01 4.96233333e+01 5.03641667e+01 1.95784442e−01 2.24654677e−01 0.44558346e−01 4.8Z1551499e+02 4.87855176e+02 4.98658107e+02]]

### Feature Tensor (Scaled Runner Profile)

[3.14433259 3.08058607 1.74315336 −0.92475758 −0.5362047 −0.90107941 −0.81146885 −1.15755997 −1.74963181 −0.45978856 −0.1082623 1.44279791]

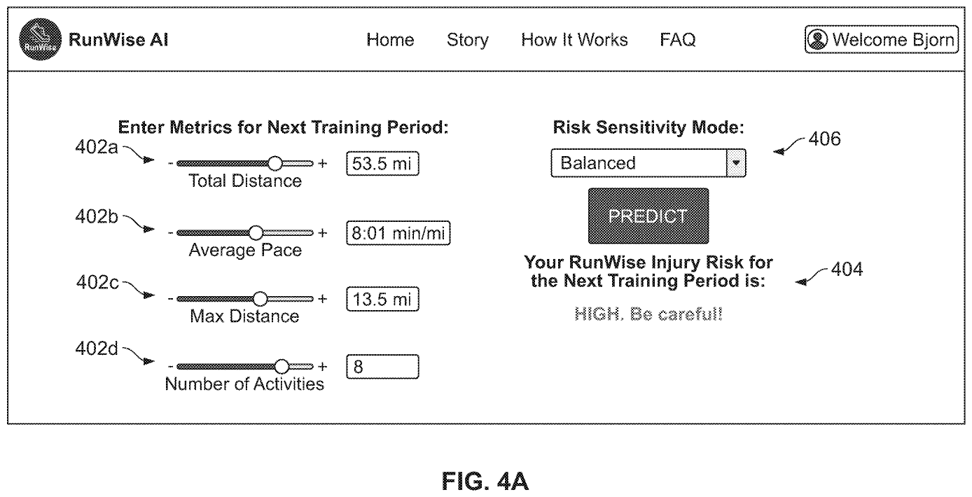

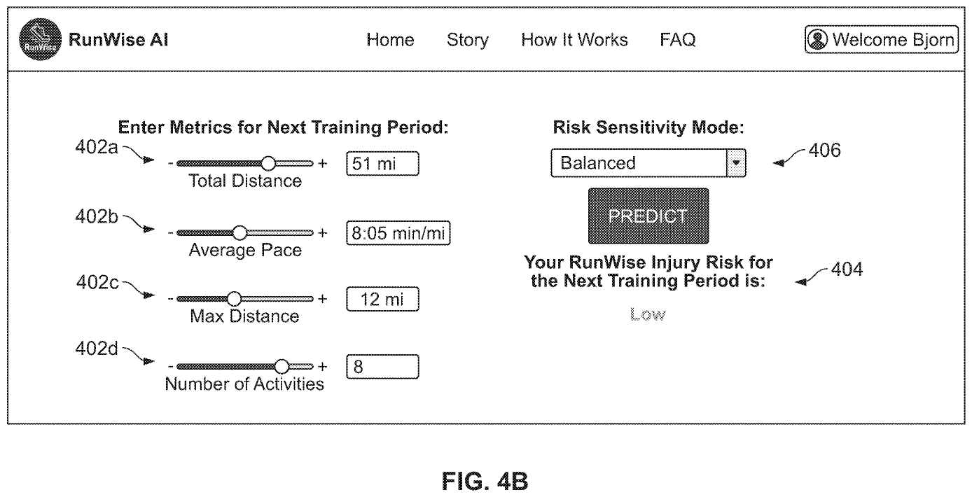

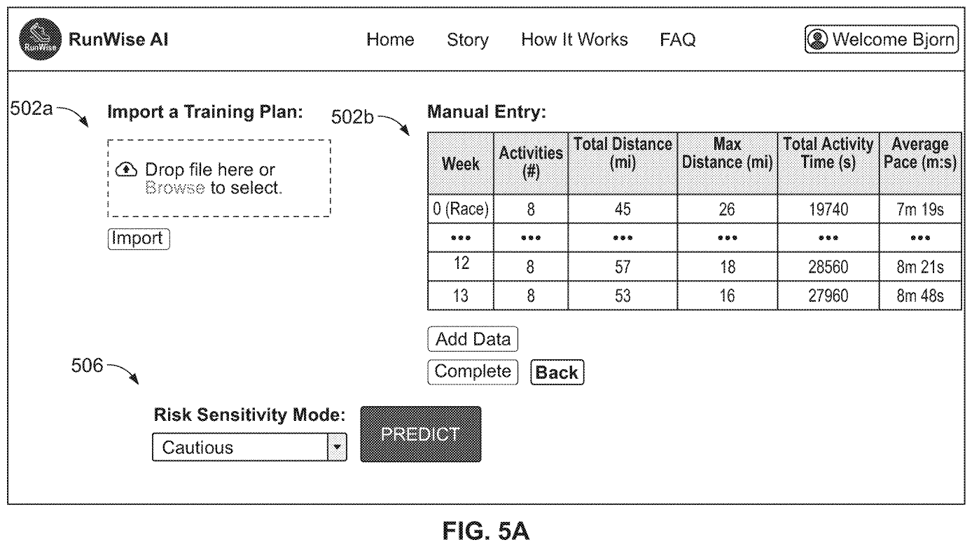

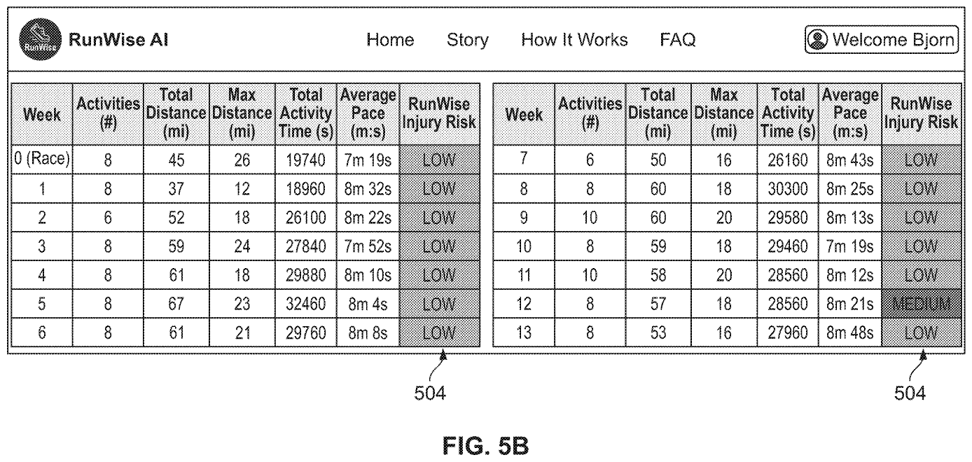

At step 230, the method includes inputting, into a trained machine learning model, the runner profile. The runner profile is a “feature tensor”, e.g. the feature tensor associated with the week of Jul. 31, 2023 shown above. In other words, the runner profile input into the trained machine learning model has been scaled. In some implementations, the runner profile input into the model at step includes metrics from a past or completed training period, for example the most-recently completed training period. A retrospective runner profile can be calculated, for example, based on the runner's historical data (volume, intensity, etc.). In other implementations, the runner profile input into the model at step includes metrics calculated for a future (e.g., the next) training period. A prospective runner profile can be calculated, for example, based on the runner's training plan (volume, intensity, etc.) for an upcoming training period (see e.g. FIGS. 4A-5B). Machine learning models trained to predict a risk of running-related injuries are described in U.S. Pat. No. 11,515,045, issued Nov. 29, 2022, titled “PREDICTING RISK OF RUNNING-RELATED INJURY USING A MACHINE LEARNING MODEL AND RELATED MACHINE LEARNING TRAINING METHODS” and U.S. Patent App. Pub. No. 2025/0054635, published Feb. 13, 2025, titled “MACHINE LEARNING-BASED METHODS AND SYSTEMS FOR PREDICTING RUNNING-RELATED INJURIES,” the disclosures of which are incorporated herein by reference in their entireties.

It should be understood that the trained machine learning model is operating in inference mode. In other words, the machine learning model has already been trained with a dataset (or “data set”). Techniques for training a machine learning model for predicting running-related injuries are described in U.S. Pat. No. 11,515,045, issued Nov. 29, 2022, titled “PREDICTING RISK OF RUNNING-RELATED INJURY USING A MACHINE LEARNING MODEL AND RELATED MACHINE LEARNING TRAINING METHODS,” the disclosure of which is incorporated herein by reference in its entirety. This disclosure contemplates that the machine learning model is a supervised learning model. According to supervised learning, the machine learning model “learns” a function that maps an input (sometimes referred to herein as the “features”) to an output (sometimes referred to herein as the “target”) based on a data set, which includes a plurality of samples from a running-related training dataset tagged with one or more labels (e.g., the injury/no injury tags), during model training mode. It should be understood that supervised learning is provided only as an example. This disclosure contemplates that the machine learning model may be a semi-supervised learning model in some implementations. Semi-supervised learning models are trained with a data set including both labeled data as well as unlabeled data.

The machine learning model can be an artificial neural network. Optionally, the machine learning model is a deep neural network, which includes multiple hidden layers between the input and output layers (described below). An artificial neural network is a computing system including a plurality of interconnected neurons (e.g., also referred to as “nodes”). This disclosure contemplates that the nodes can be implemented using a computing device (e.g., a processing unit and memory as described herein). The nodes can optionally be arranged in a plurality of layers such as input layer, output layer, and one or more hidden layers. Each node is connected to one or more other nodes in the artificial neural network. For example, each layer has a plurality of nodes, where each node is connected to all nodes in the previous layer. The nodes in a given layer are not interconnected with one another, i.e., the nodes in a given layer function independently of one another. As used herein, nodes in the input layer receive data (sometimes referred to herein as the “features” or input) from outside of the artificial neural network, nodes in the hidden layer(s) modify the data between the input and output layers, and nodes in the output layer provide the results (sometimes referred to herein as the “target” or output).

Each node in the artificial neural network is configured to receive an input and implement a function (sometimes referred to herein as the “activation function”). In other words, the activation function defines the node output for a given input. Activation functions include, but are not limited to, binary step, sigmoid, tanh, and rectified linear unit (ReLU). Additionally, each node is associated with a respective weight. Artificial neural networks are trained with a data set to minimize or maximize an objective function, which is a measure of the artificial neural network's performance. The objective function may be a cost function. Cost functions include, but are not limited to, mean squared error (MSE), mean absolute error, L1 loss (least absolute deviations), L2 loss (least squares loss), and cross-entropy loss. Training algorithms for artificial neural networks include, but are not limited to, backpropagation (BP). The training algorithm tunes the node weights and/or bias to minimize or maximize the objective function. For example, BP involves computing the gradient of the objective function with respect to the respective weights for each of the nodes. It should be understood that any algorithm that finds the minimum or maximum of the objective function can be used to for training an artificial neural network. Although artificial neural networks are provided as an example, this disclosure contemplates that the machine learning model can be other types of models including, but not limited to, a logistic regression model or a support vector machine.

At step 240, the method includes predicting, using the trained machine learning model, a risk of a musculoskeletal injury for the user. As described above, the machine learning model is trained to map the input to the output. In the examples described herein, the input is the runner profile, and the output is the risk of musculoskeletal injury, e.g., running-related musculoskeletal injury. The runner profile includes one or more “features” that are input into the machine learning model, which predicts risk of musculoskeletal injury based on the features. The risk of musculoskeletal injury is therefore the “target” of the machine learning model. In other words, the trained machine learning model is configured to analyze input “features” and predict risk of musculoskeletal injury based on the same. In some implementations, the trained machine learning model outputs a probability of musculoskeletal injury. Alternatively, the trained machine learning model classifies the runner profile into a plurality of risk categories. Risk categories can optionally include injury/no injury, low risk/high risk, low risk/medium risk/high risk, etc. classifications. As described herein, the musculoskeletal injury is a running-related injury such as an injury affecting the runner's bones, joints, or soft tissue and also force the runner to rest for an extended time period.

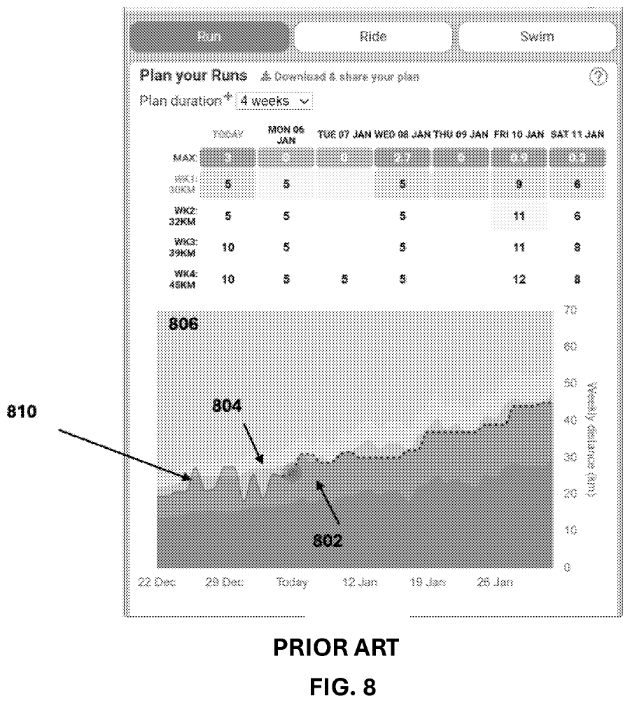

At step 250, the method includes outputting the risk of musculoskeletal injury. The risk of musculoskeletal injury is configured for display on a user interface of an application. As described above, the application can be a fitness tracking platform. Optionally, the risk of musculoskeletal injury is output to an API of the fitness tracking platform, and the fitness tracking platform integrates the risk of musculoskeletal injury into its native display. Alternatively, the application can be a running-related application, and the running-related application can integrate the risk of musculoskeletal injury into its native display. Thus, the method can further include displaying, on the user interface, the risk of musculoskeletal injury. The user interface can optionally include one or more display elements for displaying the risk of musculoskeletal injury. Displaying the risk of musculoskeletal injury on the user interface assists the runner in understanding injury risk, and optionally allows the runner to assess, adjust, tailor, etc. his training schedule to minimize likelihood of, or in some cases avoid, suffering a musculoskeletal injury. This is in contrast to displays according to existing techniques. For example, the impulse response model plot of FIG. 7 fails to display, and much less provide, an injury risk. And, even if the graphical display of ACR plot in FIG. 8 is interpreted as an injury risk, it is neither based on machine learning nor does it include display element as described herein.

Optionally, when the model predicts medium or high risk, the method can further include providing a user interface including one or more input elements that allow the runner to adjust his future training schedule. As described below with regard to FIGS. 4A-4B, such input elements may include text boxes and/or sliders. For example, the input elements can allow the runner to adjust the volume and/or intensity of a future planned run (e.g. tomorrow's planned run). Alternatively or additionally, the input elements can allow the runner to adjust the total volume, maximum volume, intensity, and/or number of runs in an upcoming training period (e.g. a subsequent week). Steps 240 and 250 can be repeated (and optionally repeated iteratively) responsive to adjustments, for example until the runner is satisfied that risk has been reduced or eliminated.

Alternatively, when the model predicts medium or high risk, the method can further include automatically adjusting the runner's training future training plan to reduce or minimize risk. In contrast to receiving inputs via a user interface, the runner's future training plan (e.g. volume and/or intensity) for one or more future planned runs can be adjusted without user input until risk is reduced or eliminated. In other words, this disclosure contemplates automatically providing the runner with a training plan designed to reduce or eliminate risk.

The methods of presentation described herein facilitate training plan adjustment to reduce injury risk. For example, in some implementations, the method optionally includes adjusting a training plan for a runner based on the risk of musculoskeletal injury. This may include one or more of: reducing the number of planned runs in the next training period, reducing the planned volume in the next training period, reducing the planned intensity in the next training period, and/or reducing the planned long run volume in the next training period. The visualization on the user interface aids the runner in making such adjustments. Optionally, the method includes performing the method of FIG. 2A after the adjustment. In other words, this disclosure contemplates an iterative process to identify a training plan that minimizes the likelihood of musculoskeletal injury. As described below, this adjustment may be performed using one of the user interfaces shown in FIGS. 4A-5B.

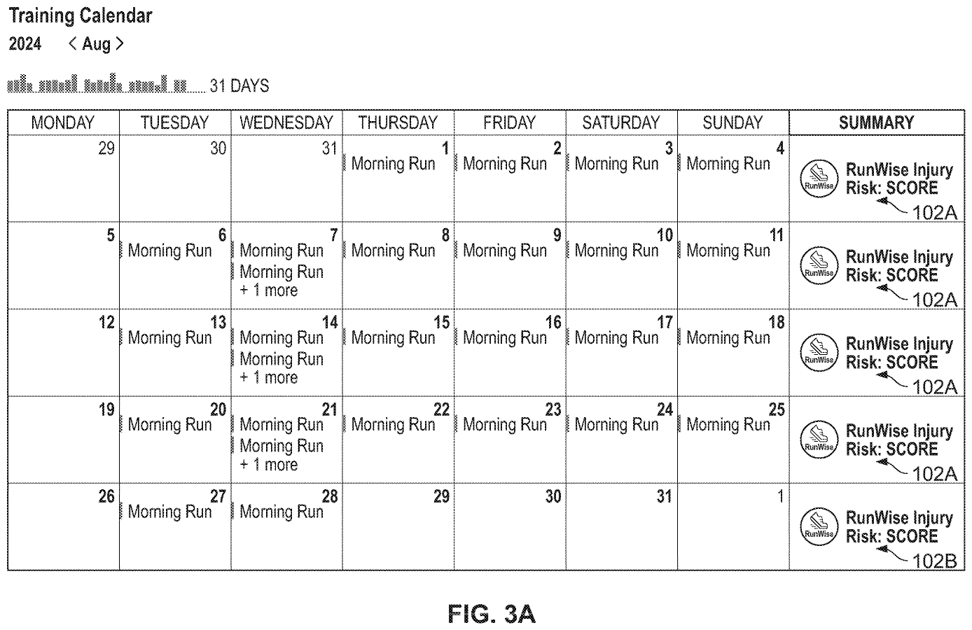

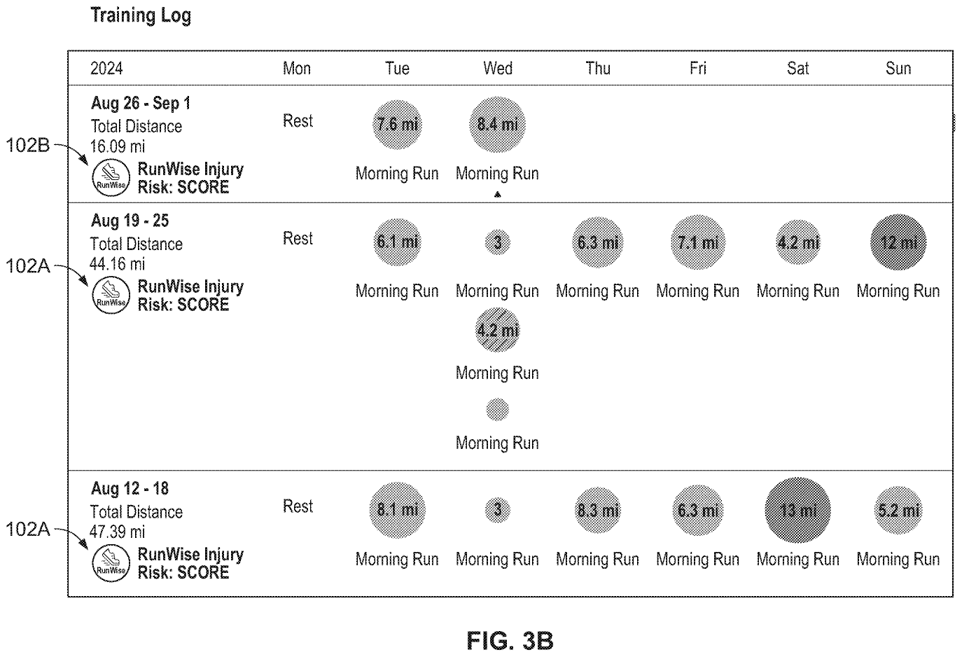

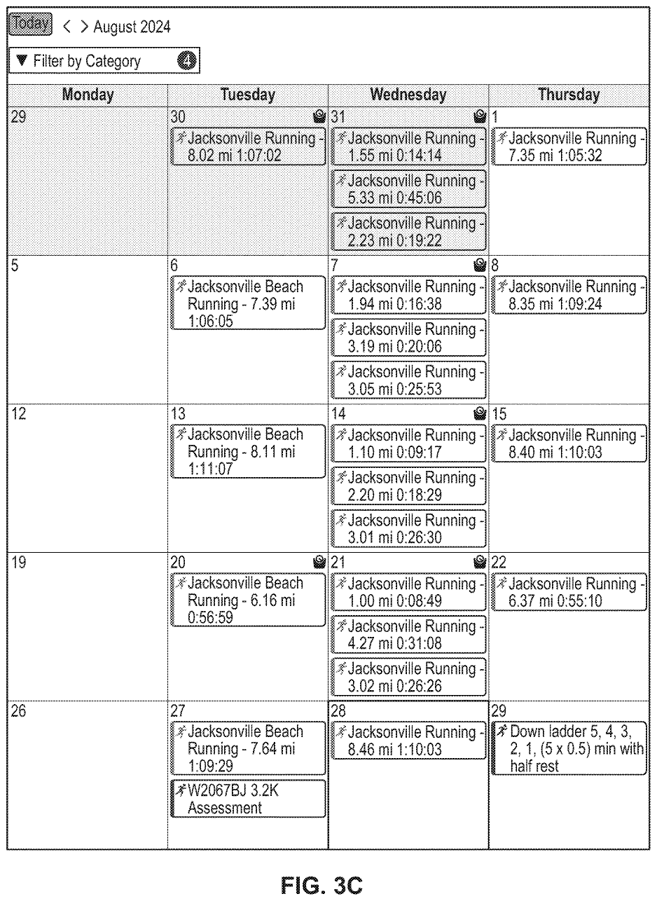

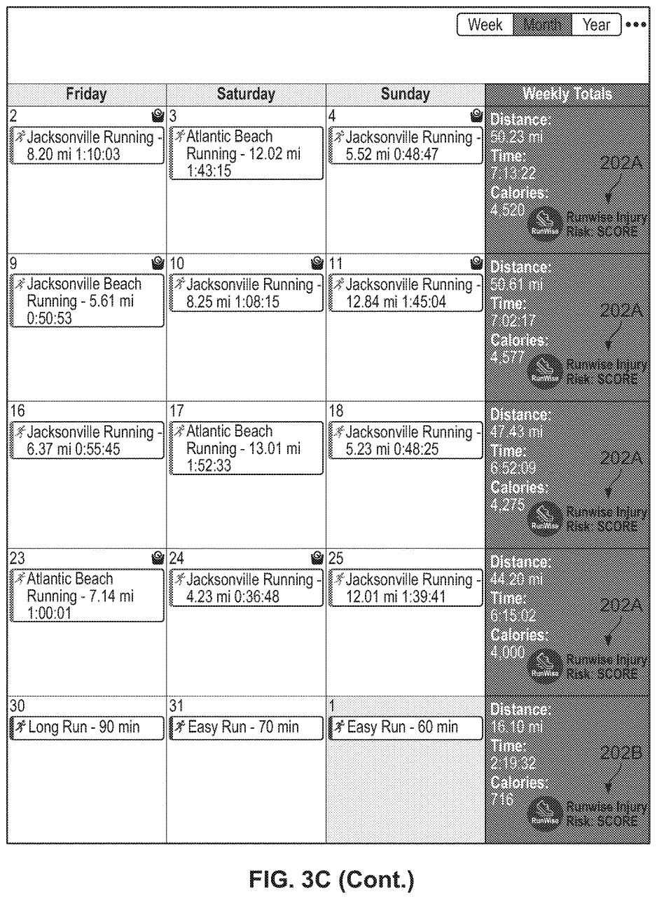

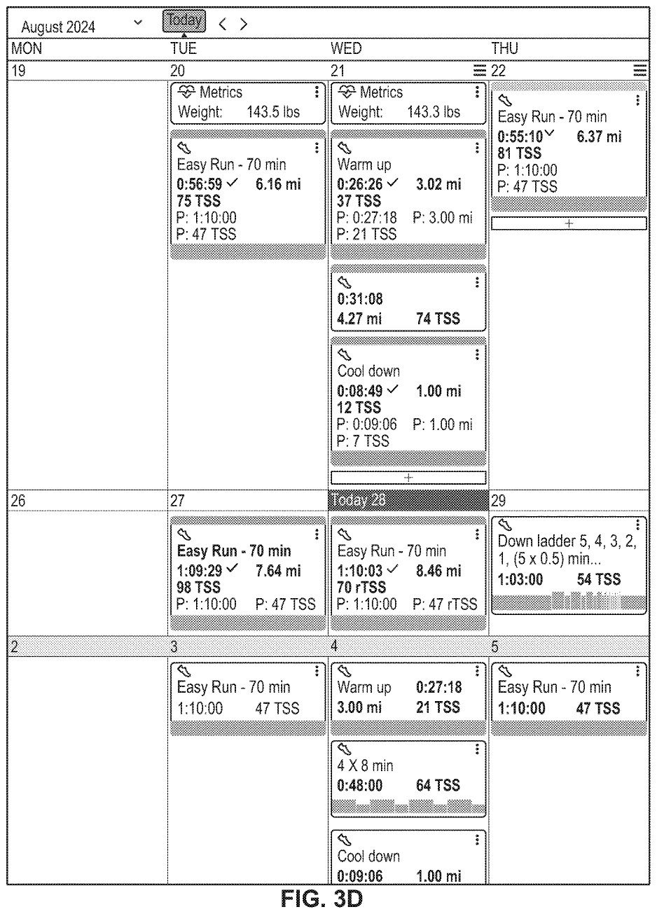

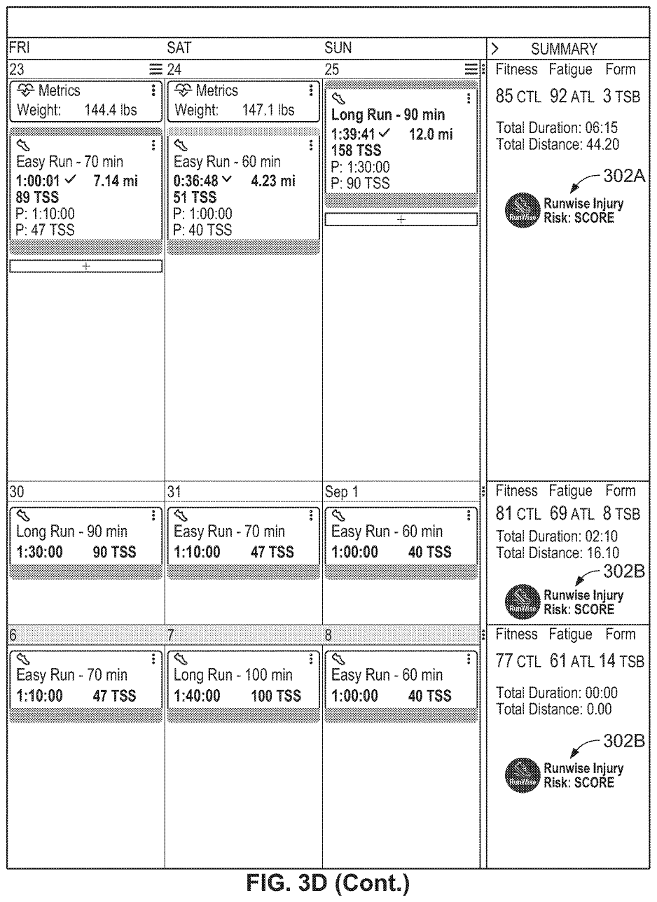

In some implementations, the user interface includes a journal view of the user's training data. As used herein, a journal view is a layout format that correlates time periods (e.g. days, weeks, months, etc.) with training activities. The training activities may be completed (i.e. historical or past activities) and/or planned (i.e. future activities). Optionally, the journal view may be a grid view. As used herein, a grid view is a layout format that displays content in a structured, two-dimensional arrangement of rows and columns. It is used in user interfaces to present data, images, or items in an organized, visually aligned way. Each item in the grid occupies a “cell” within the structure, and all cells may share consistent dimensions, creating a clean and uniform appearance. For example, the grid view may be a calendar. Example user interfaces including display of the risk of musculoskeletal injury on a calendar or training log are shown in FIGS. 3A-3D. Training calendars and logs are known in the art. For example, the STRAVA app and website, the GARMIN CONNECT app and website, and the TRAININGPEAKS app and website offer calendar and/or log views of a runner's training data, including both historical (i.e. completed) and planned (i.e. future) activities. Conventional tools like these, however, do not provide information about risk of musculoskeletal injury as described herein. FIGS. 3A-3D illustrate examples where such risk information is displayed on training calendar and log views. As described below, respective risks of musculoskeletal injury are displayed in relation to the journal view of training data.

FIG. 3A illustrates a training calendar view according to one aspect of this disclosure. Respective risks of musculoskeletal injury 102A, 102B are displayed adjacent to each week in the calendar. The calendar view was captured on Aug. 28, 2024, so the weeks beginning July 29, August 5, August 12, and August 19 include retrospective training data. Accordingly, the risk of musculoskeletal injury 102A displayed adjacent to each of the weeks beginning July 29, August 5, August 12, and August 19 were predicted based on retrospective training data. This means that the predictions were made based on the runner's historical data, i.e., the retrospective training data is associated with running that was completed previously. On the other hand, the week beginning August 26 is in progress. The entries on August 27 and August 28 represent historical training data, i.e., these runs were completed previously, while runs for August 29-September 1 have yet to be completed (i.e., entries are blank). Mondays (August 5, 12, 19, and 26) are rest days (i.e. no running activities). Accordingly, the risk of musculoskeletal injury 102B displayed adjacent to the week beginning August 26 was predicted based, at least partially, on prospective training plan data (not shown in FIG. 3A). Specifically, the risk of musculoskeletal injury 102B displayed adjacent to the week beginning August 26 was predicted based on the combination of historical data (August 27-28) and training plans (August 29-September 1). This means that the prediction was made based on information including the runner's future training plan, i.e., the prospective training plan data is associated with running that is planned to be completed in the future.

FIG. 3B illustrates a training log view according to one aspect of this disclosure. Respective risks of musculoskeletal injury 102A, 102B are displayed adjacent to each week in the log. The training log was captured on Aug. 28, 2024, so the weeks beginning August 12 and August 19 include retrospective training data. Accordingly, the risk of musculoskeletal injury 102A displayed adjacent to each of the weeks beginning August 12 and August 19 were predicted based on retrospective training data, i.e., the predictions were made based on the runner's historical data. On the other hand, the week beginning August 26 is in progress. The entries on August 27 and August 28 represent historical training data, i.e., these runs were completed previously, while runs for August 29-September 1 have yet to be completed (i.e., entries are blank). Mondays (August 12, 19, and 26) are rest days (i.e. no running activities). Accordingly, the risk of musculoskeletal injury 102B displayed adjacent to the week beginning August 26 was predicted based, at least partially, on prospective training plan data (not shown in FIG. 3B). Specifically, the risk of musculoskeletal injury 102B displayed adjacent to the week beginning August 26 was predicted based on the combination of historical data (August 27-28) and training plans (August 29-September 1), i.e., the prediction was made based on information including the runner's future training plan.

FIG. 3C illustrates a calendar view according to another aspect of this disclosure. Respective risks of musculoskeletal injury 202A, 202B are displayed adjacent to each week in the calendar. The calendar was captured on Aug. 28, 2024, so the weeks beginning July 29, August 5, August 12, and August 19 include retrospective training data. Accordingly, the risk of musculoskeletal injury 202A displayed adjacent to each of the weeks beginning July 29, August 5, August 12, and August 19 were predicted based on retrospective training data, i.e., the predictions were made based on the runner's historical data. On the other hand, the week beginning August 26 is in progress. The entries on August 27 and August 28 represent historical training data, i.e., these runs were completed previously, while the entries for August 29-September 1 represent training plan data. Mondays (July 29 and August 5, 12, 19, and 26) are rest days (i.e. no running activities). Specifically, the risk of musculoskeletal injury 202B displayed adjacent to the week beginning August 26 was predicted based on the combination of historical data (August 27-28) and training plans (August 29-September 1). The entries “Down ladder 5, 4, 3, 2, 1, (5×0.5) min with half rest,” “Long Run—90 min,” “Easy Run—70 min,” and “Easy Run—60 min” on August 29, August 30, August 31, and September 1, respectively, represent prescribed training for those days, i.e., these runs are planned in the future but not yet completed. Accordingly, the risk of musculoskeletal injury 202B displayed adjacent to the week beginning August 26 was predicted based, at least partially, on prospective training plan data, i.e., the prediction was made based on information including the runner's future training plan.

FIG. 3D illustrates a calendar view according to yet another aspect of this disclosure. Respective risks of musculoskeletal injury 302A, 302B are displayed adjacent to each week in the calendar. The calendar was captured on Aug. 28, 2024, so the week beginning August 19 includes retrospective training data. Accordingly, the risk of musculoskeletal injury 302A displayed adjacent to the week beginning August 19 was predicted based on retrospective training data, i.e., the predictions were made based on the runner's historical data. On the other hand, the week beginning August 26 is in progress, and the week beginning September 2 is in the future. Specifically, the entries on August 27 and August 28 represent historical training data, i.e., these runs were completed previously, while the entries for August 29-September 8 represent training plan data, i.e., these runs are planned in the future. Mondays (August 19 and 26 and September 2) are rest days (i.e. no running activities). Specifically, the risk of musculoskeletal injury 302B displayed adjacent to the week beginning August 26 was predicted based on the combination of historical data (August 27-28) and training plans (August 29-September 1). The entries “Down ladder 5, 4, 3, 2, 1, (5×0.5) min with half rest,” “Long Run—90 min,” “Easy Run—70 min,” and “Easy Run—60 min” on August 29, August 30, August 31, and September 1, respectively, represent prescribed training for those days. Accordingly, the risk of musculoskeletal injury 302B displayed adjacent to the week beginning August 26 was predicted based, at least partially, on prospective training plan data, i.e., the prediction was made based on information including the runner's future training plan. Additionally, the risk of musculoskeletal injury 302B displayed adjacent to the week beginning September 2 was predicted based entirely on training plans (September 2-September 8). The entries “Easy Run—70 min,” “Warm up . . . 4×8 min . . . Cool down,” “Easy Run—70 min,” “Easy Run—70 min,” “Long Run—100 min,” and “Easy Run—60 min” on September 3, September 4, September 5, September 6, September 7, September 8, respectively, represent prescribed training for those days. Accordingly, the risk of musculoskeletal injury 302B displayed adjacent to the week beginning September 2 was predicted based, at least partially, on prospective training plan data, i.e., the prediction was made based on information including the runner's future training plan.

In some aspects, the techniques described herein relate to a computer-implemented method for presenting risk of running-related injury to a user, including: receiving running-related data for a user (see e.g. FIG. 2A, step 210); creating a runner profile for the user based on the running-related data (see e.g. FIG. 2A, step 220); inputting the runner profile into a trained machine learning model (see e.g. FIG. 2A, step 230); predicting, using the trained machine learning model, a risk of musculoskeletal injury for the user (see e.g. FIG. 2A, step 240); and displaying, on a user interface, the risk of musculoskeletal injury, wherein the user interface comprises a journal view of the user's training data (see e.g. FIGS. 3A-3D). This disclosure contemplates that the steps above can be performed by a fitness tracking platform. In such implementation, there may be no need to communicate data via an API of the fitness tracking platform, for example when the fitness tracking platform hosts the runner's training data.

With reference to FIG. 2B, an example method and user interface for presenting predicted running-related injury risk is described. This disclosure contemplates that the method of FIG. 2B can be performed using one or more computing devices, e.g., computing device 600 shown in FIG. 6. The user interface provides input elements through which a user enters prospective training plan data, referred to as first running-related data. This first running-related data is combined with historical training data received from a connected application, referred to as second running-related data, to construct a runner profile comprising features for input into a trained machine learning model. The machine learning model processes the runner profile and outputs a predicted risk of musculoskeletal injury, which is displayed to the user on the user interface. The user interface further enables iterative adjustment of the prospective training plan, with updated risk predictions displayed in response to each adjustment, allowing the user to evaluate the injury risk implications of different training scenarios before committing to a plan.

The disclosed user interface provides a technical improvement over prior art training load displays by enabling a runner to interact with a machine learning-based injury risk prediction system through a structured input and display mechanism. Unlike impulse response model plots, which provide no injury risk information, or ACR displays, which reduce injury risk to a single dimensionless ratio, the disclosed interface presents a machine learning-derived injury risk prediction that reflects complex, multidimensional patterns in training data. The iterative input and display capability further provides a technical benefit by allowing a user to systematically explore the relationship between prospective training parameters and predicted injury risk, a relationship that is not intuitively apparent and cannot be derived from existing training load visualizations. This enables runners to make prospective, data-informed adjustments to planned training in order to reduce injury risk before it is incurred.

At step 260, the method includes providing a user interface including one or more input elements for collecting first running-related data for a user. The first running-related data is associated with a training plan. The first running-related data is therefore prospective data, i.e. the user has not yet completed these running activities. As used herein, an input element can include form control (e.g., a user interface (UI) element that allows user input such as checkboxes, dropdowns, etc.), input field (e.g., a UI that allows user to enter data such as text boxes), or UI control (e.g., an interactive UI element such as buttons and sliders).

An example user interface including input elements 402a, 402b, 402c, 402d (sometimes referred to herein individually as “input element 402” and collectively as “input elements 402”) is shown in FIGS. 4A-4B. The user interface of this implementation facilitates planning for a future training period, for example next week (e.g. the next 7-day period). The input elements in FIGS. 4A-4B include a total volume field 402a and an intensity field 402b, which correspond to the total mileage and average pace planned for the future training period. The input elements also include a maximum volume field 402c, which corresponds to the maximum daily mileage planned for the future training period. It should be understood that such information can be used to calculate a long run fraction for the future training period. The input elements further include a consistency field 402d, which corresponds to the number of running activities planned for the future training period. Optionally, the input elements can further include a dynamic field or a physiological field (not shown in FIGS. 4A-4B). Additionally, in FIGS. 4A-4B, the input elements 402 include text boxes and sliders. While both input field and UI control elements are shown in FIGS. 4A-4B, it should be understood that either would be sufficient to collect the first running-related data. It should also be understood that the input elements 402 shown in FIGS. 4A-4B are provided only as examples. This disclosure contemplates providing a user interface with more, fewer, and/or different input elements than shown in the figures. As a non-limiting example, a user interface that facilitates planning for tomorrow (e.g. a single day) can include a volume field and an intensity field.

At step 262, the method includes receiving the first running-related data, where the first running-related data is entered at the user interface. In FIG. 4A, the user entered the following first running-related data: 53.5 miles, 8:01 min/mi, 13.5 mi, and 8 activities in input elements 402a, 402b, 402c, and 402d, respectively. This represents the user's primary training plan for the future training period (e.g., next week). In FIG. 4B, the user entered the following first running-related data: 51 miles, 8:05 min/mi, 12 mi, and 8 activities in input elements 402a, 402b, 402c, and 402d, respectively. This represents the user's alternative training plan for the future training period (e.g., next week). As compared to the primary training plan, the alternative training plan is lower volume (51 mi vs. 53.5 mi) with a shorter long run (12 mi vs. 13.5 mi). It is also at a slower pace (8:05 min/mi vs. 8:01 min/mi).

At step 264, the method includes receiving, from an application, second running related data for the user. As described above, the connected application can optionally be a fitness tracking platform such as the STRAVA app and website, the GARMIN CONNECT app and website, the TRAININGPEAKS app and website, or the FINAL SURGE app and website. In contrast to the first running-related data, which represents the user's future training plans, the second running-related data can be historical running data, which has been stored by the fitness tracking platform. The historical running data covers a period of time of sufficient length to allow for calculation of the short-term, medium-term, and long-term metrics described herein. Optionally, the second running-related data is received via an API of the fitness tracking platform. Optionally, the second running-related data is stored in a database upon receipt via the API.

At step 266, the method includes creating a runner profile for the user based on the first and second running-related data. The runner profile is therefore based on the combination of the user's future training plan data (also referred to as “prospective training plan data”) and historical training data (also referred to as “retrospective training data”). Creation of a runner profile is discussed above in detail with regard to FIG. 2A, step 220.

At step 268, the method includes inputting the runner profile into a trained machine learning model. Input of the runner profile into the trained machine learning model is discussed above in detail with regard to FIG. 2A, step 230.

At step 270, the method includes predicting, using the trained machine learning model, a risk of musculoskeletal injury for the user. Prediction by the trained machine learning model is discussed above in detail with regard to FIG. 2A, step 240.

At step 272, the method includes displaying, on the user interface, the risk of musculoskeletal injury. For example, an example user interface including a display element 404 for displaying the risk of musculoskeletal injury is shown in FIGS. 4A-4B. The risk of musculoskeletal injury in FIG. 4A is HIGH, while the risk of musculoskeletal injury in FIG. 4B is LOW. As discussed above, there are differences between the primary training plan (entered in user interface of FIG. 4A) and the alternative training plan (entered in user interface of FIG. 4B). These differences result in a reduction in risk of musculoskeletal injury.

Displaying the risk of musculoskeletal injury on the user interface facilitates the runner in understanding injury risk, and optionally allows the runner to assess, adjust, tailor, etc. his training schedule to minimize likelihood of, or in some cases avoid, suffering a musculoskeletal injury. This is in contrast to displays according to existing techniques. For example, the impulse response model plot of FIG. 7 fails to display, and much less provide, an injury risk. And, even if the graphical display of ACR plot in FIG. 8 is interpreted as an injury risk, it is neither based on machine learning nor does it include display element as described herein.

Optionally, in some implementations, the method includes adjusting a training plan for a runner based on the risk of musculoskeletal injury. This may include one or more of: reducing the number of planned runs in the next training period, reducing the planned volume in the next training period, reducing the planned intensity in the next training period, and/or reducing the planned long run volume in the next training period. This can be performed in an iterative fashion. For example, in the example shown in FIG. 4A, the user entered the following first running-related data: 53.5 miles, 8:01 min/mi, 13.5 mi, and 8 activities in input elements 402a, 402b, 402c, and 402d, respectively at steps 260-262. Thereafter, as a result of steps 264-272, the risk of musculoskeletal injury displayed at display element 404 is HIGH in FIG. 4A. Thus, the user made adjustments to the training plan in an effort to decrease injury risk. In particular, in the example shown in FIG. 4B, the user entered the following first running-related data: 51 miles, 8:05 min/mi, 12 mi, and 8 activities in input elements 402a, 402b, 402c, and 402d, respectively at steps 260-262. In other words, the user reduced planned total and long run volumes, as well as decreased planned intensity. Thereafter, as a result of steps 264-272, the risk of musculoskeletal injury displayed at display element 404 is LOW in FIG. 4B. By adjusting the training plan, the user reduced risk of musculoskeletal injury from HIGH to LOW. The user interface facilitates the user's ability to iterate (e.g. using the input elements and PREDICT button), which is particularly beneficial because it is difficult to understand which feature, features, and/or combination of features contribute to injury risk. The trained machine learning model is configured to find complex patterns in the data. Thus, the ability to iterate adjustments, with visual feedback, provides the user with a solution to a problem. It should be understood that FIGS. 4A-4B, which illustrate a single iteration to reach LOW risk, are provided only as an example. It should be understood that a plurality of iterations may be needed.

Referring now to FIGS. 5A-5B, another user interface including input elements for collecting running-related data and display elements for displaying risk are shown. The user interface of FIGS. 5A-5B can be used for assessing training plans. For example, a marathon training plan can include instructions for a 12-16 week period or even longer. This disclosure contemplates that the user interface of FIG. 5A-5B can be used with the method of FIG. 2B in a similar manner as the user interface of FIGS. 4A-4B. For example, the user interface of FIG. 5A includes input elements 502a, 502b (referred to individually as “input element 502” and collectively as “input elements 502”). Input element 502a is a window for importing a training plan. The user can drag and drop the training plan file or search for the training plan file. Optionally, the training plan file is a file type for storing tabular data such as CSV or XLSX file format. Optionally, the training plan file is a portable document file (PDF) from which data can be extracted. Input element 502b, on the other hand, includes input fields for manual entry of training plan data. Input elements 502a and/or 502b can be used at steps 260-262 of FIG. 2B, e.g. for collecting the first running-related data, which is the training plan data in the examples. Additionally, the user interface of FIG. 5B includes display elements 504 for displaying the risk of musculoskeletal injury. In FIG. 5B, the risk is displayed in a journal view, e.g., a respective risk is displayed adjacent to different weeks in the training plan. It should also be understood that the elements shown in FIGS. 5A-5B are provided only as examples. This disclosure contemplates providing a user interface with more, fewer, and/or different elements than shown in the figures.

In some implementations, the user interface can optionally further include an input element for collecting a risk sensitivity mode. For example, FIGS. 4A-4B include risk sensitivity mode input element 406, and FIG. 5A includes risk sensitivity mode input element 506. Non-limiting risk sensitivity modes include Cautious, Balanced, and Strict. In FIGS. 4A-4B and 5A, the input element is a dropdown, which shows Cautious. Optionally, risk sensitivity mode Cautious corresponds to a model with high recall (or specificity), while risk sensitivity mode Strict corresponds to a model with high precision (or sensitivity). Risk sensitivity mode Balanced is between modes Cautious and Strict. Precision and recall are two complementary metrics in machine learning classification that measure different aspects of a model's performance. Precision and recall pull against each other. For example, if the classification threshold is lowered to catch more positives, recall goes up but precision tends to drop because the model will also flag more false positives. Raising the threshold does the opposite.

The user may desire a model with high recall in some circumstances, e.g., when training for a goal race. Recall is a measure of how many actual positive cases were correctly identified:

$$Recall = \frac{True Positivies ( TP )}{True Positives ( TP ) + False Negatives ( FN )} .$$

Accordingly, high recall results in fewer missed detections. In the context of running-related injury prediction, high recall may be desirable when the cost of missing detection of high injury risk (i.e. a true positive) is costly. A model with high recall catches more injury-risk runner profiles but may incorrectly flag some low-risk runner profiles as MEDIUM or HIGH risk. This may be desirable in the lead up to a goal race, when the margin for error is small. Sustaining an injury in the lead up to a goal race can have devastating consequences for the runner, which may include not being able to compete. Thus, a missed warning (false negative) could mean the runner trains through a risky period and gets hurt. A false alarm (false positive) just prompts the runner to be a bit more cautious that week, which is a lower-cost error.

On the other hand, the user may desire a model with high precision in some circumstances, e.g., when training to stay fit. Precision is a measure of how many predicted positive cases are actually positive:

$$Precision = \frac{True Positivies ( TP )}{True Positives ( TP ) + False Positives ( FP )} .$$

A high-precision model is more conservative with warnings, but may miss some genuine risk signals. In the context of running-related injury prediction, high precision may be desirable when false positives are costly or otherwise unwanted. A model with high precision might miss true risks but will have fewer false alarms. This may be desirable during general fitness training to minimize unnecessary alarm and/or training interruptions at times when the runner tolerates these risks.

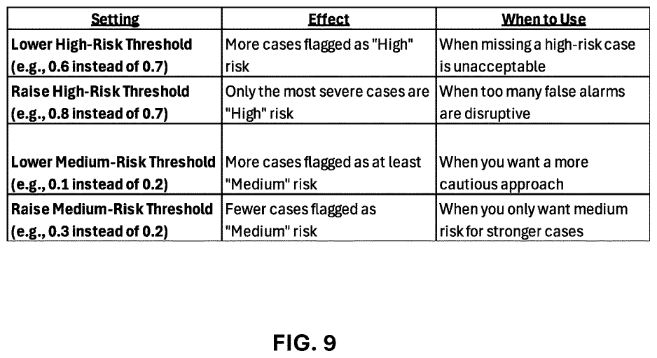

The method optionally further includes receiving the risk sensitivity mode, wherein the risk sensitivity mode is entered at the user interface. The risk sensitivity mode chosen by the user can be used to select an appropriate model, e.g., high-recall model (or Cautious), high-precision model (or Strict), or balanced model (or Balanced). In other words, the platform can deploy a plurality of models from which the user can choose based on its performance characteristics. Alternatively or additionally, the risk sensitivity mode selected by the user can be used to alter the model's classification thresholds. In this implementation, there may be a single or multiple deployed models. For example, in some implementations, the trained machine learning model described above is a classifier, where thresholds are used to control classification boundaries (e.g. LOW, MEDIUM, HIGH risk of musculoskeletal injury). Risk sensitivity modes such as Cautious, Balanced, and Strict can be presented to the user, e.g., via the risk sensitivity mode input element, and the different modes can be associated with different thresholds. For example, assuming threshold values of 0.2 and 0.7 separating LOW/MEDIUM classes and MEDIUM/HIGH classes, respectively, adjusting thresholds results in the following shown by the table of FIG. 9. It should be understood that threshold values and adjustments are provided only as examples.

Conventional injury risk assessment tools apply fixed models (e.g. CTL, ATL, TSB) or heuristics (e.g. ACR) with no mechanism for a user to calibrate them to their individual training context. The present disclosure addresses this limitation by providing a risk sensitivity mode input element through which a user selects a mode, such as Cautious, Balanced, or Strict, that adjusts the behavior of the underlying machine learning classifier. Specifically, the selected mode controls classification thresholds or selects among models optimized for different precision-recall tradeoffs, allowing the user to tune the rate of false positives against the rate of false negatives based on the cost of each error type in their current training context. For example, a runner in the final weeks before a goal race may select Cautious mode, which favors high recall and minimizes missed injury risk detections at the cost of occasional false alarms, whereas a runner in a general fitness phase may select Strict mode, which favors high precision and minimizes unnecessary alarm and/or training interruptions at the cost of occasionally missing a true risk signal. This user-driven calibration of classifier behavior through a user interface input element provides a technical improvement over fixed assessments by enabling the prediction system to be tuned to the asymmetric and context-dependent costs of prediction errors of individual runners.

In some aspects, the techniques described herein relate to a computer-implemented method, including: receiving running-related data for a user; creating a runner profile for the user based on the running-related data; receiving a risk sensitivity mode; selecting a trained machine learning model or one or more thresholds for the trained machine learning model based on the risk sensitivity mode; inputting the runner profile into a trained machine learning model; and predicting, using the trained machine learning model, a risk of musculoskeletal injury for the user. Optionally, the method further includes displaying, on a user interface, the risk of musculoskeletal injury.

In some implementations, the method further includes generating a report that summarizes the user's training data. Optionally, the report includes the risk of musculoskeletal injury. Optionally, the method further includes displaying, on the user interface, the report. Alternatively or additionally, the method optionally further includes transmitting the report to the user. The report can optionally be a text file or PDF. For example, the report can be transmitted to the user by email or text message. Such a report may be useful if the user desires to share training data with a coach, medical professional, or other third party. This may be particularly desirable if the user has suffered an injury and desires advice on recovery.

In some implementations, the user interface further includes an input element for collecting an indication of an injury. The method further includes receiving the indication of the injury, wherein the indication is entered at the user interface. Optionally, the indication includes a type, a description, or a location of the injury, such as “left knee pain,” “shin splints,” or “Achilles tendinopathy.” This disclosure contemplates that such information can be used for labeling a dataset for training purposes, for example by associating the injury indication with the runner profile and training data logged at or preceding the time of injury.

In some implementations, the method further includes: inputting, into a language model, one or more of the runner profile and the indication; generating, using the language model, a recommendation about the injury; and outputting the recommendation, wherein the recommendation is configured for display on the user interface. For example, the language model can generate a recommendation regarding training modifications, recovery strategies, or when to seek professional evaluation. The present disclosure contemplates applying known language models in the context of running-related injury risk management. Non-limiting examples of language models include ChatGPT, available from OpenAI of San Francisco, California; Claude, available from Anthropic of San Francisco, California; and Gemini, available from Google Inc. of Mountain View, California. In some implementations, the method further includes: receiving a user input including a question concerning the injury; processing, by a language model, the user input to extract key information; generating, by the language model, a response based on the key information; and outputting the response, wherein the response is configured for display on the user interface. For example, a user may submit a natural language query such as “is it safe to run tomorrow given my current risk level and knee pain?” and receive a contextually relevant response generated by the language model based on the extracted key information. The present disclosure contemplates applying known language models in the context of running-related injury risk and training management. Non-limiting examples of language models include ChatGPT, available from OpenAI of San Francisco, California; Claude, available from Anthropic of San Francisco, California; and Gemini, available from Google Inc. of Mountain View, California.

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 600 upon which the methods described herein may be implemented is illustrated. It should be understood that the example computing device 600 is only one example of a suitable computing environment upon which the methods described herein may be implemented. Optionally, the computing device 600 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 600 typically includes at least one processing unit 606 and system memory 604. Depending on the exact configuration and type of computing device, system memory 604 may be volatile (such as random access memory (RAM)), non-volatile (such as read-only memory (ROM), flash memory, etc.), or some combination of the two. This most basic configuration is illustrated in FIG. 6 by box 602. The processing unit 606 may be a standard programmable processor that performs arithmetic and logic operations necessary for operation of the computing device 600. The computing device 600 may also include a bus or other communication mechanism for communicating information among various components of the computing device 600.

Computing device 600 may have additional features/functionality. For example, computing device 600 may include additional storage such as removable storage 608 and non-removable storage 610 including, but not limited to, magnetic or optical disks or tapes. Computing device 600 may also contain network connection(s) 616 that allow the device to communicate with other devices. Computing device 600 may also have input device(s) 614 such as a keyboard, mouse, touch screen, etc. Output device(s) 612 such as a display, speakers, printer, etc. may also be included. The additional devices may be connected to the bus in order to facilitate communication of data among the components of the computing device 600. All these devices are well known in the art and need not be discussed at length here.

The processing unit 606 may be configured to execute program code encoded in tangible, computer-readable media. Tangible, computer-readable media refers to any media that is capable of providing data that causes the computing device 600 (i.e., a machine) to operate in a particular fashion. Various computer-readable media may be utilized to provide instructions to the processing unit 606 for execution. Example tangible, computer-readable media may include, but is not limited to, volatile media, non-volatile media, removable media and non-removable media implemented in any method or technology for storage of information such as computer readable instructions, data structures, program modules or other data. System memory 604, removable storage 608, and non-removable storage 610 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 606 may execute program code stored in the system memory 604. For example, the bus may carry data to the system memory 604, from which the processing unit 606 receives and executes instructions. The data received by the system memory 604 may optionally be stored on the removable storage 608 or the non-removable storage 610 before or after execution by the processing unit 606.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language, and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1-5. (canceled)

6. A computer-implemented method for presenting risk of running-related injury to a user, comprising:
providing a user interface comprising one or more input elements for collecting first running-related data for a user;
receiving the first running-related data, wherein the first running-related data is entered at the user interface;
receiving, from an application, second running related data for the user;
creating a runner profile for the user based on the first and second running-related data;
inputting the runner profile into a trained machine learning model;
predicting, using the trained machine learning model, a risk of musculoskeletal injury for the user; and
displaying, on the user interface, the risk of musculoskeletal injury.

7. The computer-implemented method of claim 6, wherein the user interface further comprises one or more display elements for displaying the risk of musculoskeletal injury.

8. The computer-implemented method of claim 6, wherein the one or more input elements include a total volume field and an intensity field.

9. The computer-implemented method of claim 8, wherein the one or more input elements further include a maximum volume field.

10. The computer-implemented method of claim 8, wherein the one or more input elements further include a consistency field.

11. The computer-implemented method of claim 8, wherein the one or more input elements further include a dynamic field or a physiological field.

12. The computer-implemented method of claim 6, wherein the user interface further comprises an input element for collecting a risk sensitivity mode, the method further comprising receiving the risk sensitivity mode, wherein the risk sensitivity mode is entered at the user interface.

13. The computer-implemented method of claim 6, further comprising generating a report that summarizes the user's training data.

14. The computer-implemented method of claim 13, further comprising displaying, on the user interface, the report.

15. The computer-implemented method of claim 13, further comprising transmitting the report to the user.

16. The computer-implemented method of claim 13, wherein the report comprises the risk of musculoskeletal injury.

17. The computer-implemented method of claim 6, wherein the user interface further comprises an input element for collecting an indication of an injury, the method further comprising receiving the indication, wherein the indication is entered at the user interface.

18. The computer-implemented method of claim 17, wherein the indication comprises a type, a description, or a location of the injury.

19. The computer-implemented method of claim 17, further comprising:
inputting, into a language model, one or more of the runner profile and the indication;
generating, using the language model, a recommendation about the injury; and
outputting the recommendation, wherein the recommendation is configured for display on the user interface.

20. The computer-implemented method of claim 17, further comprising:
receiving a user input comprising a question concerning the injury;
processing, by a language model, the user input to extract key information;
generating, by the language model, a response based on the key information; and
outputting the response, wherein the response is configured for display on the user interface.

21-26. (canceled)
